SEQUENCE LISTING (7 sequences)

SEQ ID NO: 1 - protein, 750 aa, Saccharomyces cerevisiae
MDRIAQQYRNGKRDNNGNRMASSAISEKGHIQVNQTRTPGQMPVYRGETINLSNLPQNQIKPCKDLDDVNIRRNNSNRHSKILLLDLCAGPNTNSFLGNTNAKDITVLSLPLPSTLVKRSNYPFENLLKNYLGSDEKYIEFTKIIKDYDIFIFSDSFSRISSCLKTTFCLIEKFKKFICHFFPSPYLKFFLLEGSLNDSKAPSLGKNKKNCILPKLDLNLNVNLTSRSTLNLRINIPPPNDSNKIFLQSLKKDLIHYSPNSLQKFFQFNMPADLAPNDTILPNWLKFCSVKENEKVILKKLFNNFETLENFEMQRLEKCLKFKKKPLHQKQLSQKQRGPQSTDDSKLYSLTSLQRQYKSSLKSNIQKNQKLKLIIPKNNTSSSPSPLSSDDTIMSPINDYELTEGIQSFTKNRYSNILPYEHSRVKLPHSPKPPAVSEASTTETKTDKSYPMCPVDAKNHSCKPNDYINANYLKLTQINPDFKYIATQAPLPSTMDDFWKVITLNKVKVIISLNSDDELNLRKWDIYWNNLSYSNHTIKLQNTWENICNINGCVLRVFQVKKTAPQNDNISQDCDLPHNGDLTSITMAVSEPFIVYQLQYKNWLDSCGVDMNDIIKLHKVKNSLLFNPQSFITSLEKDVCKPDLIDDNNSELHLDTANSSPLLVHCSAGCGRTGVFVTLDFLLSILSPTTNHSNKIDVWNMTQDLIFIIVNELRKQRISMVQNLTQYIACYEALLNYFALQKQIKNALPC

SEQ ID NO: 2 - protein, 281 aa, Saccharomyces cerevisiae
MSNHSEILERPETPYDITYRVGVAENKNSKFRRTMEDVHTYVKNFASRLDWGYFAVFDGHAGIQASKWCGKHLHTIIEQNILADETRDVRDVLNDSFLAIDEEINTKLVGNSGCTAAVCVLRWELPDSVSDDSMDLAQHQRKLYTANVGDSRIVLFRNGNSIRLTYDHKASDTLEMQRVEQAGGLIMKSRVNGMLAVTRSLGDKFFDSLVVGSPFTTSVEITSEDKFLILACDGLWDVIDDQDACELIKDITEPNEAAKVLVRYALENGTTDNVTVMVVFL

SEQ ID NO: 3 - DNA, 2253 bp, Saccharomyces cerevisiae
atggatcgcatagcacagcaatatcgtaatggcaaaagagacaataacggcaatagaatggcttcttccgctatatcggaaaagggccacatacaagtcaatcaaactagaacacctggtcaaatgcccgtctatagaggtgaaactataaatctgtctaaccttccccaaaatcaaattaaaccgtgcaaagatttggacgacgttaacatacggcggaacaactctaataggcattctaaaatacttttactagatctgtgcgctggccccaataccaactcatttttaggcaataccaatgctaaggatatcacagttttatcgttgccgctacccagcactttggtgaaaaggtcgaactacccgttcgagaacttactaaagaattaccttggatctgatgaaaagtatattgagttcacaaagatcatcaaagattatgatattttcattttcagtgattcgtttagcagaatttcgagttgtttaaaaacaactttttgcctcattgagaagtttaaaaagttcatctgccatttttttccatctccttatttgaaattctttcttctcgaaggctctctgaatgatagcaaggccccctcatcaggaaaaaataagaaaaattgcatcttgcccaaattggatttgaacttgaatgtaaacttaacttcaaggtcaactttaaatttaagaataaacatacctccacccaatgattcaaataaaatatttttacagtctctgaaaaaggatttaattcattattctcctaattctttgcaaaagtttttccaattcaatatgcctgctgacttagcacctaacgacacgattttaccgaattggctaaaattctgctccgtaaaagaaaatgaaaaggtaatattaaagaaactctttaacaattttgaaactttagaaaattttgaaatgcaaagattagagaaatgcctgaaattcaagaaaaagcctttacatcaaaagcagctatcacaaaagcagaggggtccgcaatccacggatgattcaaaattatattctttaactagtttgcaacgacagtataaaagttctttgaaaagcaacatacagaaaaatcaaaacctaaaattaattataccaaaaaacaacacatcttcttcgccatcaccattatcttccgatgatactataatgacaccaataaatgattacgaacttactgaaggaattcagtcttttactaagaatagatattctaatatcttaccttacgaacattcaagagtaaagttacctcactccccgaaaccacctgcagtttctgaagcatccacaaccgaaactaaaacagataagtcatatccgatgtgtcccgtagatgcaaaaaaccactcctgcaaaccgaacgactatatcaatgcgaactatttgaagctcacgcaaattaatcctgatttcaagtatattgctacccaagctccgcttccttctacgatggatgatttttggaaggttattactttaaataaagttaaagtaataatatcattgaattctgacgatgaattgaatttaagaaaatgggatatttactggaataatctatcatattccaaccacactatcaaacttcaaaacacctgggagaatatttgcaatattaacggctgtgttctcagagtctttcaagtcaagaaaacagctccacaaaatgataatatcagtcaagattgtgaccttccgcataatggtgaccttacttccactaccatggctgtatccgagccgtttattgtttaccaattacaatacaagaattggttagattcatgcggcgtagatatgaatgacatcattaaactacacaaagtcaaaaattcgttattgtttaacccgcaaagttttattacaagcctcgaaaaggatgtttgcaagcctgatttgatagatgataataatagtgatttacatctcgatacagcaaattcatcgccgctattagtccattgttctgcagggtgtggaagaacaggtgttttcgttaccttggatttcctgctaagtattctttcacctacaacaaatcactcaaataagattgatgtttggaatatgactcaggaccttatctttatcatagtgaatgaattaagaaagcaaaggatttcaatggtacagaatctaactcaatatatcgcttgttatgaggcattattaaattattttgccctgcaaaagcagataaagaacgcgttaccttgttaa

SEQ ID NO: 4 - DNA, 846 bp, Saccharomyces cerevisiae
atgagtaatcattctgaaatcttagaaaggccagaaacaccatatgacataacttatagagtaggtgtggcggaaaataaaaactcgaaatttcggaggacaatggaagatgttcatacgtatgttaaaaactttgcttcaagattagattggggatatttcgcggtgtttgatggacatgctgggattcaggcctccaaatggtgtggtaaacatcttcatacaattatagagcaaaacattttggcagatgaaacacgagatgttagagatgtattgaacgattcattcctagccattgacgaagaaattaatacaaaacttgtaggaaatagtggatgtactgctgctgtttgcgtattacgttgggagcttccggattcagtttctgatgattcaatggatttagcccaacaccaaagaaagttatatacagcaaatgttggtgattctcgaatagtattgtttagaaacgggaacagcataagactgacttatgatcataaagcatctgacactttggagatgcagagagttgaacaagcaggtggcctgataatgaaaagtcgtgtaaatggtatgctggcggtgacgagatcgttaggggataaattttttgatagtttagtagtgggcagcccatttaccacgagcgtagaaataacttctgaggacaaatttttaatcctagcgtgtgatggattatgggatgttattgatgatcaagatgcatgcgaattaatcaaggatattactgaacctaatgaagctgcaaaagtcttggttagatatgctttggaaaatggcacaacagataatgtaacggtcatggttgtcttcctctaa

SEQ ID NO: 5 - DNA, 457 bp, Artificial Sequence
  Synthetic
gacgatgaggaagaggaggctgcgtttgacgacgaagaggatgataatgaggaagaagaagaagaagaggacgcggatgaagagaacgcctctcgtctaagaaatttaaaaagagaaggagcagcaatgtacagagaagaggaagaagaagaaaaagataggagcgagacaaaaagaagaagggttgcggtcatcgaggacgacgaagacgaggattagaggagacgttactttgtttatatatattagtatgtacaatcgcaaagaaatggagtgatgacatgttgtagtatttagtatgaggttactgtgtgggagattttaccatgatttttggcgagaacacgccatgaaatgtctttgtacgaaaatcattacccgcattaatattttttttctttttaaagctcagttgaccctttctcattcccttcttaaaacaactgtgtgatccttg

SEQ ID NO: 6 - DNA, 1000 bp, Artificial Sequence
  Synthetic
gttaattcaaattaattgatatagttttttaatgagtattgaatctgtttagaaataatggaatattatttttatttatttatttatattattggtcggctcttttcttctgaaggtcaatgacaaaatgatatgaaggaaataatgatttctaaaattttacaacgtaagatatttttacaaaagcctagctcatcttttgtcatgcactattttactcacgcttgaaattaacggccagtccactgcggagtcatttcaaagtcatcctaatcgatctatcgtttttgatagctcattttggagttcgcgattgtcttctgttattcacaactgttttaatttttatttcattctggaactcttcgagttctttgtaaagtctttcatagtagcttactttatcctccaacatatttaacttcatgtcaatttcggctcttaaattttccacatcatcaagttcaacatcatcttttaacttgaatttattctctagctcttccaaccaagcctcattgctccttgatttactggtgaaaagtgatacactttgcgcgcaatccaggtcaaaactttcctgcaaagaattcaccaatttctcgacatcatagtacaatttgttttgttctcccatcacaatttaatatacctgatggattcttatgaagcgctgggtaatggacgtgtcactctacttcgcctttttccctactccttttagtacggaagacaatgctaataaataagagggtaataataatattattaatcggcaaaaaagattaaacgccaagcgtttaattatcagaaagcaaacgtcgtaccaatccttgaatgcttcccaattgtatattaagagtcatcacagcaacatattcttgttattaaattaattattattgatttttgatattgtataaaaaaaccaaatatgtataaaaaaagtgaataaaaaataccaagtatggagaaatatattagaagtctatacgttaaa

SEQ ID NO: 7 - DNA, 2820 bp, Saccharomyces cerevisiae
taggtgacaaacgcctagccgccggagcctgccggtaccggcttggcttcagttgctgatctcggcgcggaaaaatcagcgccccacgccaaaaggttcgtattttttctttttttttctaatcttccatctattcggtagcgatgattcatttctctgaaaaaaaaaaaaaaaaaaaaaaatgaaaaagaatatttttttgatgaacttgtatttctcttatctggttgatatatatgctatcatttattttcttatcaagtttccaaatttctaatcctttctccaccatccctaattaataattcagatctacgtcacaccgtaatttgtattgtttttttccttcattgtctaaaaccgaagaattcatcagccacagttactagttcatttgaagcgaaattacacacattttccctgttacaatagaaagtattttacaaaatctaaaccctttgagctaagaggagataaatacaacagaatcaattttcaaatgatcagacaatctacgctaaaaaacttcgctattaagcgttgctttcaacatatagcataccgcaacacacctgccatgagatcagtagctctcgcgcagcgcttttatagttcgtcttcccgttattacagtgcgtctccattaccagcctctaaaaggccagagcctgctccaagtttcaatgttgatccattagaacagcccgctgaaccttcaaaattggctaagaaactacgcgctgagcctgacatggatacctctttcgtcggtttaactggtggtcaaatatttaacgaaatgatgtccagacaaaacgttgatactgtatttggttatccaggtggtgctatcctacctgtttacgatgccattcataacagtgataaattcaacttcgttcttccaaaacacgaacaaggtgccggtcacatggcagaaggctacgccagagcttctggtaaaccaggtgttgtcttggttacttctgggccaggtgccaccaatgtcgttactccaatggcagatgcctttgcagacgggattccaatggttgtctttacagggcaagtctcaactagtgctatcggtactgatgctttccaagaggctgacgtcgttggtatttctagatcttgtacgaaatggaatgtcatggtcaagtccgtggaagaattgccattgcgtattaacgaggcttttgaaattgccacgagcggtagaccgggaccagtcttggtcgatttaccaaaggatgttacagcagctatcttaagaaatccaattccaacaaaaacaactcttccatcaaacgcactaaaccaattaaccagtcgcgcacaagatgaatttgtcatgcaaagtatcaataaagcagcagatttgatcaacttggcaaagaaacctgtcttatacgtcggtgctggtattttaaaccatgcagatggtccaagattactaaaagaattaagtgaccgtgctcaaatacctgtcaccactactttacaaggtttaggttcattcgaccaagaagatccaaaatcattggatatgcttggtatgcacggttgtgctactgccaacctggcagtgcaaaatgccgacttgataattgcagttggtgctagattcgacgaccgtgtcactggtaatatttctaaattcgctccagaagctcgtcgtgcagctgccgagggtagaggtggtattattcatttcgaggttagtccaaaaaacataaacaaggttgttcaaactcaaatagcagtggaaggtgatgctacgaccaatctgggcaaaatgatgtcaaagattttcccagttaaggagaggtctgaatggtttgctcaaataaataaatggaagaaggaatacccatacgcttatatggaggagactccaggatctaaaattaaaccacagacggttataaagaaactatccaaggttgccaacgacacaggaagacatgtcattgttacaacgggtgtggggcaacatcaaatgtgggctgctcaacactggacatggagaaatccacatactttcatcacatcaggtggtttaggtacgatgggttacggtctccctgccgccatcggtgctcaagttgcaaagccagaatctttggttattgacattgatggtgacgcatcctttaacatgactctaacggaattgagttctgccgttcaagctggtactccagtgaagattttgattttgaacaatgaagagcaaggtatggttactcaatggcaatccctgttctacgaacatcgttattcccacacacatcaattgaaccctgatttcataaaactagcggaggctatgggtttaaaaggtttaagagtcaagaagcaagaggaattggacgctaagttgaaagaattcgtttctaccaagggcccagttttgcttgaagtggaagttgataaaaaagttcctgttttgccaatggtggcaggtggtagcggtctagacgagttcataaattttgacccagaagttgaaagacaacagactgaattacgtcataagcgtacaggcggtaagcactgaatttcaaaaacatttatttcaaaagcattttcagtaaaaaatgcagactttattattatttaatcgtgcttcttatatatgacattctaccaaatcggtagtcatgtatatttttttcgtatatactttatatatttttttctaaaaaactaatgacggctaaaattaagtcatagatgaataataagttcaattcaagtgagttggtagtatttgataaatctcgcgccgaagctgaagtgcaaggattgataatg

(19) United States
(12) Patent Application Publication
(10) Pub. No.: US 2021/0147792 A1
(43) Pub. Date: May 20, 2021

(54) YEAST OVER-EXPRESSING PROTEIN PHOSPHATASES ASSOCIATED WITH THE HOG PATHWAY

(71) Applicant: DANISCO US INC., Palo Alto, CA (US)

(72) Inventor: Andrei Miasnikov, Union City, CA (US)

(21) Appl. No.: 17/046,920

(22) PCT Filed: Apr. 11, 2019
(86) PCT No.: PCT/US19/26990

§ 371 Date: Oct. 12, 2020

Related U.S. Application Data
(60) Provisional application No. 62/656,684, filed on Apr. 12, 2018.

(51) Int. Cl. C12N 1/16; C12N 9/16

(52) U.S. Cl. CPC C12N 1/16; C12N 9/16; C12Y 301/03016; C12Y 301/03048; C12N 2510/00

(57) ABSTRACT

Described are compositions and methods relating to modified yeast that over-express protein phosphatases associated with the HOG pathway. The yeast produces an increased amount of ethanol and deceased amount of glycerol compared to parental cells. Such yeast is particularly useful for large-scale ethanol production from starch substrates.

### TECHNICAL FIELD

The present compositions and methods relate to modified yeast cells that over-expresses protein phosphatases associated with the HOG pathway. The yeast cells produce an increased amount of ethanol and a deceased amount of glycerol compared to their parental cells. Such yeast is particularly useful for large-scale ethanol production from starch substrates.

### BACKGROUND

Yeast-based ethanol production is based on the conversion of sugars into ethanol. The current annual fuel ethanol production by this method is about 90 billion liters worldwide. It is estimated that about 70% of the cost of ethanol production is the feedstock. Since the ethanol production volume is so large, even small yield improvements have massive economic impact for the industry. The conversion of one mole of glucose into two moles of ethanol and two moles of carbon dioxide is redox-neutral, with the maximum theoretical yield being about 51%. The current industrial yield is about 45%; therefore, there are opportunities to increase ethanol production. Despite advances in yeast productivity, the need exists to further modify yeast metabolic pathways to maximize ethanol production, while not increasing the production of undesirable by-products.

### SUMMARY

The present compositions and methods relate to modified yeast that over-express protein phosphatases associated with the HOG pathway. Aspects and embodiments of the compositions and methods are described in the following, independently-numbered, paragraphs.

1. In a first aspect, modified yeast cells derived from parental yeast cells are provided, the modified cells comprising a genetic alteration that causes the modified cells to produce an increased amount of protein phosphatase polypeptides associated with the high-osmolarity glycerol pathway compared to the parental cells, wherein the modified cells produce during fermentation an increased amount of ethanol and/or a decreased amount of glycerol compared to the amount of ethanol and glycerol produced by the parental cells under equivalent fermentation conditions.

2. In some embodiments of the modified cells of paragraph 1, the genetic alteration comprises the introduction into the parental cells of a nucleic acid capable of directing the expression of a protein phosphatase polypeptide to a level above that of the parental cell grown under equivalent conditions.

3. In some embodiments of the modified cells of paragraph 1, the genetic alteration comprises the introduction of an expression cassette for expressing a protein phosphatase polypeptide.

4. In some embodiments of the modified cells of any of paragraphs 1-3, the protein phosphatase is PTC1 or PTP2.

5. In some embodiments of the modified cells of any of paragraphs 1-4, the cells further comprising one or more genes of the phosphoketolase pathway.

6. In some embodiments of the modified cells of paragraph 5, the genes of the phosphoketolase pathway are selected from the group consisting of phosphoketolase, phosphotransacetylase and acetylating acetyl dehydrogenase.

7. In some embodiments of the modified cells of any of paragraphs 1-6, the amount of increase in the expression of the protein phosphatase polypeptide is at least about 200% compared to the level expression in the parental cells grown under equivalent conditions.

8. In some embodiments of the modified cells of paragraphs 1-7, the amount of increase in the production of mRNA encoding the protein phosphatase polypeptide is at least about 400% compared to the level in the parental cells grown under equivalent conditions.

9. In some embodiments of the modified cells of any of paragraphs 1-8, the cells further comprise an exogenous gene encoding a carbohydrate processing enzyme.

10. In some embodiments, the modified cells of any of paragraphs 1-9 further comprise an alteration in the glycerol pathway and/or the acetyl-CoA pathway.

11. In some embodiments, the modified cells of any of paragraphs 1-10 further comprise an alternative pathway for making ethanol.

12. In some embodiments of the modified cells of any of paragraphs 1-11, the cells are of a Saccharomyces spp.

13. In another aspect, a method for decreasing the production of acetate from yeast cells grown on a carbohydrate substrate is provided, comprising: introducing into parental yeast cells a genetic alteration that increases the production of protein phosphatase polypeptides compared to the amount produced in the parental cells.

14. In some embodiments of the modified cells of paragraph 13, the cells having the introduced genetic alteration are the modified cells are the cells of any of paragraphs 1-12.

15. In some embodiments of the modified cells of paragraph 13 or 14, the decrease in acetate production is at least 10%, at least 15%, at least 20%, or at least 25%.

16. In some embodiments of the modified cells of paragraphs 13-15, protein phosphatase polypeptides are over-expressed by at least 200%.

17. In some embodiments of the modified cells of any of paragraphs 12-16, protein phosphatase polypeptides are over-expressed by at least 15-fold.

18. In some embodiments of the modified cells of any of paragraphs 12-17, the carbohydrate substrate is present in an industrial medium.

These and other aspects and embodiments of present modified cells and methods will be apparent from the description, including the Drawings/Figures.

### BRIEF DESCRIPTION OF THE DRAWINGS

FIG. 1 depicts a plasmid map of an expression vectors used to express PTC1.

FIG. 2 depicts a plasmid map of an expression vectors used to express PTP2.

FIG. 3 shown the amount of ethanol produced by modified cells with a 95% confidence interval.

FIG. 4 shown the amount of glycerol produced by modified cells with a 95% confidence interval.

### DETAILED DESCRIPTION

### I. Definitions

Prior to describing the present yeast and methods in detail, the following terms are defined for clarity. Terms not defined should be accorded their ordinary meanings as used in the relevant art.

As used herein, the term “alcohol” refers to an organic compound in which a hydroxyl functional group (—OH) is bound to a saturated carbon atom.

As used herein, the terms “yeast cells,” “yeast strains,” or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “engineered yeast cells,” “variant yeast cells,” “modified yeast cells,” or similar phrases, refer to yeast that include genetic modifications and characteristics described herein. Variant/modified yeast do not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins,” or “homologs.” Such proteins can be derived from organisms of different genera and/or species, or different classes of organisms (e.g., bacteria and fungi), or artificially designed. Related proteins also encompass homologs determined by primary sequence analysis, determined by secondary or tertiary structure analysis, or determined by immunological cross-reactivity, or determined by their functions.

As used herein, the term “homologous protein” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′5, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype. The term “allele” is generally preferred when an organism contains more than one similar genes, in which case each different similar gene is referred to as a distinct “allele.”

As used herein, “constitutive” expression refers to the production of a polypeptide encoded by a particular gene under essentially all typical growth conditions, as opposed to “conditional” expression, which requires the presence of a particular substrate, temperature, or the like to induce or activate expression.

As used herein, the term “expressing a polypeptide” and similar terms refers to the cellular process of producing a polypeptide using the translation machinery (e.g., ribosomes) of the cell.

As used herein, “over-expressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes a promoter, and amino acid coding region and a terminator (i.e., promoter::amino acid coding region::terminator) and other nucleic acid sequence needed to allow the encoded polypeptide to be produced in a cell. Expression cassettes can be exogenous (i.e., introduced into a cell) or endogenous (i.e., extant in a cell).

As used herein, the terms “fused” and “fusion” with respect to two DNA fragments, such as a promoter and the coding region of a polypeptide refer to a physical linkage causing the two DNA fragments to become a single molecule.

As used herein, the terms “wild-type” and “native” are used interchangeably and refer to genes, proteins or strains found in nature, or that are not intentionally modified for the advantage of the presently described yeast.

As used herein, the term “protein of interest” refers to a polypeptide that is desired to be expressed in modified yeast. Such a protein can be an enzyme, a substrate-binding protein, a surface-active protein, a structural protein, a selectable marker, a signal transducer, a receptor, a transporter, a transcription factor, a translation factor, a co-factor, or the like, and can be expressed. The protein of interest is encoded by an endogenous gene or a heterologous gene (i.e., gene of interest”) relative to the parental strain. The protein of interest can be expressed intracellularly or as a secreted protein.

As used herein, “disruption of a gene” refers broadly to any genetic or chemical manipulation, i.e., mutation, that substantially prevents a cell from producing a function gene product, e.g., a protein, in a host cell. Exemplary methods of disruption include complete or partial deletion of any portion of a gene, including a polypeptide-coding sequence, a promoter, an enhancer, or another regulatory element, or mutagenesis of the same, where mutagenesis encompasses substitutions, insertions, deletions, inversions, and combinations and variations, thereof, any of which mutations substantially prevent the production of a function gene product. A gene can also be disrupted using CRISPR, RNAi, antisense, or any other method that abolishes gene expression. A gene can be disrupted by deletion or genetic manipulation of non-adjacent control elements. As used herein, “deletion of a gene,” refers to its removal from the genome of a host cell. Where a gene includes control elements (e.g., enhancer elements) that are not located immediately adjacent to the coding sequence of a gene, deletion of a gene refers to the deletion of the coding sequence, and optionally adjacent enhancer elements, including but not limited to, for example, promoter and/or terminator sequences, but does not require the deletion of non-adjacent control elements. Deletion of a gene also refers to the deletion a part of the coding sequence, or a part of promoter immediately or not immediately adjacent to the coding sequence, where there is no functional activity of the interested gene existed in the engineered cell.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, a “functional polypeptide/protein” is a protein that possesses an activity, such as an enzymatic activity, a binding activity, a surface-active property, a signal transducer, a receptor, a transporter, a transcription factor, a translation factor, a co-factor, or the like, and which has not been mutagenized, truncated, or otherwise modified to abolish or reduce that activity. Functional polypeptides can be thermostable or thermolabile, as specified.

As used herein, “a functional gene” is a gene capable of being used by cellular components to produce an active gene product, typically a protein. Functional genes are the antithesis of disrupted genes, which are modified such that they cannot be used by cellular components to produce an active gene product, or have a reduced ability to be used by cellular components to produce an active gene product.

As used herein, yeast cells have been “modified to prevent the production of a specified protein” if they have been genetically or chemically altered to prevent the production of a functional protein/polypeptide that exhibits an activity characteristic of the wild-type protein. Such modifications include, but are not limited to, deletion or disruption of the gene encoding the protein (as described, herein), modification of the gene such that the encoded polypeptide lacks the aforementioned activity, modification of the gene to affect post-translational processing or stability, and combinations, thereof.

As used herein, “attenuation of a pathway” or “attenuation of the flux through a pathway,” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “aerobic fermentation” refers to growth in the presence of oxygen.

As used herein, “anaerobic fermentation” refers to growth in the absence of oxygen.

As used herein, the expression “end of fermentation” refers to the stage of fermentation when the economic advantage of continuing fermentation to produce a small amount of additional alcohol is exceeded by the cost of continuing fermentation in terms of fixed and variable costs. In a more general sense, “end of fermentation” refers to the point where a fermentation will no longer produce a significant amount of additional alcohol, i.e., no more than about 1% additional alcohol, or no more substrate left for further alcohol production.

As used herein, an “industrial medium” is a yeast culture medium that (i) is derived from corn by liquefaction, (ii) in crude form has dry solids content of at least 33%, and preferably above 34%, (iii) in clarified form has DS content of at least 28%, and preferably above 29%, and (iv) contains a sufficient amount of carbohydrate to produce, with conventional yeast (such as FG), at least 135 g/l of ethanol, and preferably above 140 g/l of ethanol.

As used herein, the expression “carbon flux” refers to the rate of turnover of carbon molecules through a metabolic pathway. Carbon flux is regulated by enzymes involved in metabolic pathways, such as the pathway for glucose metabolism and the pathway for maltose metabolism.

As used herein, the singular articles “a,” “an” and “the” encompass the plural referents unless the context clearly dictates otherwise. All references cited herein are hereby incorporated by reference in their entirety. The following abbreviations/acronyms have the following meanings unless otherwise specified:
EC enzyme commission
PKL phosphoketolase
PTA phosphotransacetylase
AADH acetaldehyde dehydrogenases
ADH alcohol dehydrogenase
EtOH ethanol
AA α-amylase
GA glucoamylase
° C. degrees Centigrade
bp base pairs
DNA deoxyribonucleic acid
ds or DS dry solids
g or gm gram
g/L grams per liter
H₂O water
HOG high-osmolarity glycerol
HPLC high performance liquid chromatography
hr or h hour
kg kilogram
M molar
mg milligram
mL or ml milliliter
min minute
mM millimolar
N normal
nm nanometer
PCR polymerase chain reaction
ppm parts per million
PPP phosphoprotein phosphatase
PTC protein phosphatase 2C
PTFE polytetrafluoroethylene
PTP protein Tyr phosphatase
Δ relating to a deletion
microgram
μL and μl microliter
μM micromolar

### II. Modified Yeast Cells Having Increased Protein Phosphatase Expression

Described are modified yeast and methods having a genetic alteration that results in the production of increased amounts of protein phosphatase polypeptides compared to corresponding (i.e., otherwise-identical) parental cells. Protein phosphatases are enzymes that remove a phosphate group from the phosphorylated amino acid residue of a substrate protein. Proteins are phosphorylated predominantly on serine, threonine and tyrosine residues.

Applicants have discovered that yeast cells over-expressing protein phosphatases produce an increased amount of ethanol and a decreased amount of glycerol from a starch substrate compared to otherwise-identical parental cells. The exact mechanism of action is unknown, and the present compositions and methods are not limited by a theory; however, it is postulated that the protein phosphatases modulate phosphorylation in the high-osmolarity glycerol (HOG) pathway, which is important for protecting yeast against osmotic shock and yet lethal if engineered to be constitutive.

Protein phosphatases are involved in very many regulatory processes, most of which have nothing to do with glycerol production. There are multiple protein phosphatases families, including the phosphoprotein phosphatase (PPP) family, the protein Tyr phosphatase (PTP) family, and the protein phosphatase 2C (PTC) family. The protein phosphatases used to exemplify the present yeast and methods were PTC1 and PTP2, the disruption of which is known to result in lethal HOG hyperactivation (Maeda, T. et al. (1993) Mol. Cell. Biol. 13:5408-17 and Saito, H. and Tatebayashi, K. (2004) J. Biochem. 136:267-72). While it might be expected that overexpression of PTC1 and PTP2 would affect glycerol production, it was not expected that it would increase ethanol production. Even more unexpected is the observation that high level over-expression of PTC1 and PTP2 does not results in any noticeable toxic side effects and does not interfere with the ability of yeast to finish fermentation in the high density industrial fermentation medium (i.e., liquefact).

PTP2 is a tyrosine protein phosphatase from Saccharomyces cerevisiae S288C (Genbank Accession No. DAA10980.1; see, e.g., Goffeau, A. et al. (1996) Science 274:546 and Dujon, B. et al. (1997) Nature 387:98-102), having the amino acid sequence of SEQ ID NO: 1:

| | |
|---|---|
| 1 | MDRIAQQYRN GKRDNNGNRM ASSAISEKGH IQVNQTRTPG |
| | QMPVYRGETI NLSNLPQNQI |
| 61 | KPCKDLDDVN IRRNNSNRHS KILLLDLCAG PNTNSFLGNT |
| | NAKDITVLSL PLPSTLVKRS |
| 121 | NYPFENLLKN YLGSDEKYIE FTKIIKDYDI FIFSDSFSRI |
| | SSCLKTTFCL IEKFKKFICH |
| 181 | FFPSPYLKFF LLEGSLNDSK APSLGKNKKN CILPKLDLNL |
| | NVNLTSRSTL NLRINIPPPN |
| 241 | DSNKIFLQSL KKDLIHYSPN SLQKFFQFNM PADLAPNDTI |
| | LPNWLKFCSV KENEKVILKK |
| 301 | LFNNFETLEN FEMQRLEKCL KFKKKPLHQK QLSQKQRGPQ |
| | STDDSKLYSL TSLQRQYKSS |
| 361 | LKSNIQKNQK LKLIIPKNNT SSSPSPLSSD DTIMSPINDY |
| | ELTEGIQSFT KNRYSNILPY |
| 421 | EHSRVKLPHS PKPPAVSEAS TTETKTDKSY PMCPVDAKNH |
| | SCKPNDYINA NYLKLTQINP |
| 481 | DFKYIATQAP LPSTMDDFWK VITLNKVKVI ISLNSDDELN |
| | LRKWDIYWNN LSYSNHTIKL |
| 541 | QNTWENICNI NGCVLRVFQV KKTAPQNDNI SQDCDLPHNG |
| | DLTSITMAVS EPFIVYQLQY |
| 601 | KNWLDSCGVD MNDIIKLHKV KNSLLFNPQS FITSLEKDVC |
| | KPDLIDDNNS ELHLDTANSS |
| 661 | PLLVHCSAGC GRTGVFVTLD FLLSILSPTT NHSNKIDVWN |
| | MTQDLIFIIV NELRKQRISM |
| 721 | VQNLTQYIAC YEALLNYFAL QKQIKNALPC |

PTC1 is a type 2C protein phosphatase from S. cerevisiae S288C (Genbank Accession No. DAA11842.1; see, e.g., Goffeau, A. et al. (Id.) and Dujon, B. et al. (Id.)), having the amino acid sequence of SEQ ID NO: 2:

| | |
|---|---|
| 1 | MSNHSEILER PETPYDITYR VGVAENKNSK FRRTMEDVHT |
| | YVKNFASRLD WGYFAVFDGH |
| 61 | AGIQASKWCG KHLHTIIEQN ILADETRDVR DVLNDSFLAI |
| | DEEINTKLVG NSGCTAAVCV |
| 121 | LRWELPDSVS DDSMDLAQHQ RKLYTANVGD SRIVLFRNGN |
| | SIRLTYDHKA SDTLEMQRVE |
| 181 | QAGGLIMKSR VNGMLAVTRS LGDKFFDSLV VGSPFTTSVE |
| | ITSEDKFLIL ACDGLWDVID |
| 241 | DQDACELIKD ITEPNEAAKV LVRYALENGT TDNVTVMVVF L |

In some embodiments of the present compositions and methods, the protein phosphatase polypeptide that is over-expressed in modified yeast cells is a PTP protein phosphatase. In particular embodiments, the protein phosphatase polypeptide that is over-expressed in modified yeast cells is a PTP2 protein phosphatase. In some embodiments, the protein phosphatase polypeptide that is over-expressed is a PTC protein phosphatase. In particular embodiments, the protein phosphatase polypeptide that is over-expressed in modified yeast cells is a PTC1 protein phosphatase.

In particular embodiments of the present compositions and methods, the amino acid sequence of the protein phosphatase polypeptide that is over-expressed in modified yeast cells has at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99% identity, to SEQ ID NO: 1 or to SEQ ID NO: 2.

The increase in the amount of ethanol produced by the modified cells may be an increase of at least 0.5%, at least 1.0%, at least 1.5%, or even at least 2.0% or more, compared to the amount of ethanol produced by parental cells grown under the same conditions. The decrease in the amount of glycerol produced by the modified cells may be a decrease of at least 5%, at least 7%, at least 10%, or even at least 15% or more, compared to the amount of ethanol produced by parental cells grown under the same conditions.

The decrease in the amount of glycerol produced by the modified cells may be a decrease of at least 5%, at least 7%, at least 10%, or even at least 15% or more, compared to the amount of ethanol produced by parental cells grown under the same conditions.

The increase in the amount of protein phosphatase polypeptides produced by the modified cells may be an increase of at least 20%, at least 30%, at least 40%, at least 50%, at least 70%, at least 100%, at least 150%, at least 200%, at least 500%, at least 1000%, or more, compared to the amount of protein phosphatase polypeptides produced by parental cells grown under the same conditions. Alternatively, or additionally, the increase in the amount of protein phosphatase polypeptides produced by the modified cells may be at least 2-fold, at least 5-fold, at least 10-fold, at least 20-fold, at least 30-fold or more, compared to the amount of protein phosphatase polypeptides produced by parental cells grown under the same conditions.

The increase in the strength of the promoter used to control expression of the protein phosphatase polypeptides produced by the modified cells may be at least 3-fold, at least 6-fold, at least 15-fold, at least 50-fold, at least 100-fold, at least 200-fold, or more, compared to strength of the native promoter controlling protein phosphatase expression, based on the amount of mRNA produced. It is understood that relative promoter strength is not an exact scalar value. It can strongly depend on culture medium, fermentation time, temperature and other conditions. Values obtained from RNAseq data collected over the time course of fermentation in industrial medium are the most preferred, however, experimental and/or literature data obtained under different cultivation may also be used for recombinant promoter selection.

It should be noted that minor overexpression of protein phosphatase is not sufficient to produce the optimal increase in ethanol in yeast. Preferred overexpression levels are at least 10-fold resulting from the use of a strong promoter, such as, for example, promoters of glycolytic genes.

Preferably, increased protein phosphatase expression is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells.

In some embodiments, the present compositions and methods involve introducing into yeast cells a nucleic acid capable of directing the over-expression, or increased expression, of a protein phosphatase polypeptide. Particular methods include but are not limited to (i) introducing an exogenous expression cassette for producing the polypeptide into a host cell, optionally in addition to an endogenous expression cassette, (ii) substituting an exogenous expression cassette with an endogenous cassette that allows the production of an increased amount of the polypeptide, (iii) modifying the promoter of an endogenous expression cassette to increase expression, (iv) increase copy number of the same or different cassettes for over-expression of protein phosphatase, and/or (v) modifying any aspect of the host cell to increase the half-life of the polypeptide in the host cell.

In some embodiments, the parental cell that is modified already includes a gene of interest, such as a gene encoding a selectable marker, carbohydrate-processing enzyme, or other polypeptide. In some embodiments, a gene of introduced is subsequently introduced into the modified cells.

In some embodiments, the parental cell that is modified already includes an engineered pathway of interest, such as a PKL pathway to increases ethanol production, or any other pathway to increase alcohol production.

### III. Modified Yeast Cells Having Increased Protein Phosphatase Expression in Combination with Genes of an Exogenous PKL Pathway

Increased expression of protein phosphatase can be combined with expression of genes in the PKL pathway to reduce the production of elevated amounts of acetate that is associated with introducing an exogenous PKL pathway into yeast.

Engineered yeast cells having a heterologous PKL pathway have been previously described in WO2015148272 (Miasnikov et al.). These cells express heterologous phosphoketolase (PKL), phosphotransacetylase (PTA) and acetylating acetyl dehydrogenase (AADH), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells.

### IV. Combination of Increased Protein Phosphatase Expression with Other Mutations that Affect Alcohol and/or Glycerol Production

In some embodiments, in addition to expressing increased amounts of protein phosphatase expression, optionally in combination with the introduction an exogenous PKL pathway, the present modified yeast cells include additional modifications that affect ethanol and/or glycerol production.

The modified cells may further include mutations that result in attenuation of the native glycerol biosynthesis pathway and/or reuse glycerol pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). Methods to enhance the reuse glycerol pathway by over expression of glycerol dehydrogenase (GCY1) and dihydroxyacetone kinase (DAK1) to convert glycerol to dihydroxyacetone phosphate (Zhang et al. (2013) J Ind Microbiol Biotechnol. 40:1153-1160).

The modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This partially reduces the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like.

In some embodiments the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lacks a heterologous gene(s) encoding an acetylating acetaldehyde dehydrogenase, a pyruvate-formate lyase or both.

In some embodiments, the present modified yeast cells may further over-express a sugar transporter-like (STL1) polypeptide to increase the uptake of glycerol (see, e.g., Ferreira et al. (2005) Mol Biol Cell 16:2068-76; Dus̆ková et al. (2015) Mol Microbiol 97:541-59 and WO 2015023989 A1).

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

### V. Combination of Increased Protein Phosphatase Expression with Other Beneficial Mutations

In some embodiments, in addition to increased expression of protein phosphatase polypeptides, optionally in combination with other genetic modifications that benefit alcohol and/or glycerol production, the present modified yeast cells further include any number of additional genes of interest encoding proteins of interest. Additional genes of interest may be introduced before, during, or after genetic manipulations that result in the increased production of active HAC1 polypeptides. Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VI. Use of the Modified Yeast for Increased Alcohol Production

The present compositions and methods include methods for increasing alcohol production and/or reducing glycerol production, in fermentation reactions. Such methods are not limited to a particular fermentation process. The present engineered yeast is expected to be a “drop-in” replacement for convention yeast in any alcohol fermentation facility, whether using raw starch hydrolysis, simultaneous saccharification and fermentation, or other standard variations of conventional ethanol production. While primarily intended for fuel alcohol production, the present yeast can also be used for the production of potable alcohol, including wine and beer.

### VII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or α-amylase.

### VII. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the compositions and methods.

### Examples

### Example 1. Materials and Methods

### Liquefact Preparation:

Liquefact (corn mash slurry) was prepared by adding 600 ppm of urea, 0.124 SAPU/g ds FERMGEN™ 2.5× (acid fungal protease), 0.33 GAU/g ds variant Trichoderma glucoamylase (TrGA) and 1.46 SSCU/g ds Aspergillus kawachii α-amylase (AKAA), adjusted to a pH of 4.8.

### Microtiter Plate Assays:

Factory liquefact (corn mash slurry) was subjected to centrifugation, and the supernatant filtered through 0.45 μm (sterilizing) filter. Clarified liquefact adjusted to dry solids content of 29% (corresponding to the dry solids content of crude liquefact of about 34%) was supplemented with 5 ml/L of a 10 mg/ml solution of ergosterol in Tween 80 along with 600 mg/L urea, 0.124 SAPU/g ds acid fungal protease (i.e., FERMGEN™), 1.46 SSCU/g ds AKAA, and stored frozen until used. 0.33 GAU/g ds variant TrGA was added immediately before the start of fermentation.

For fermentation assays, 800 μl of clarified liquefact were placed into each well of a deep 96-well MTP (Simport T110-10) and fermentation started with 10 μl of inoculum. The inocula were grown for ˜24 h in SC6% synthetic medium containing yeast nitrogen base (0.67%), glucose (6%), and urea (0.2%), overnight. The deep well plates were closed with perforated ENZYSCREEN® lids. The lids were modified in such a way that the original rubber gasket was replaced with a (6-8 mm) slab of soft (Shore hardness 10) silicone rubber punctured with 26-gauge hypodermic needle over each of the 96 wells. This modification minimizes ethanol evaporation while allowing CO₂ to escape. Fermentation was conducted for 66 h at 32° C. with agitation.

### HPLC Analysis:

At the end of fermentation, yeast was separated from the cultures by centrifugation (10 min at 3,900 rpm) and aliquots of the supernatant were filtered using Corning FILTREX™ CLS3505 96 well plates. The HPLC analysis was performed using PHENOMENEX REZEX™ RFQ Fast Acid H+ columns (a tandem of 50×7.8 mm and 100×7.8 mm) at 60° C. with a 0.6 ml/min isocratic flow rate in 5 mM H₂SO₄. An Agilent G1312A liquid chromatograph with refractive index detection was used. Unless otherwise specified, all values are expressed in g/L.

### Example 2. Preparation of Protein Phosphatase Expression Cassettes

Two different protein phosphatases were over-expressed in yeast. The first was PTP2 from Saccharomyces cerevisiae S288C (Genbank Accession No. DAA10980.1) having the amino acid sequence of SEQ ID NO: 1:

| | |
|---|---|
| 1 | MDRIAQQYRN GKRDNNGNRM ASSAISEKGH IQVNQTRTPG |
| | QMPVYRGETI NLSNLPQNQI |
| 61 | KPCKDLDDVN IRRNNSNRHS KILLLDLCAG PNTNSFLGNT |
| | NAKDITVLSL PLPSTLVKRS |
| 121 | NYPFENLLKN YLGSDEKYIE FTKIIKDYDI FIFSDSFSRI |
| | SSCLKTTFCL IEKFKKFICH |
| 181 | FFPSPYLKFF LLEGSLNDSK APSLGKNKKN CILPKLDLNL |
| | NVNLTSRSTL NLRINIPPPN |
| 241 | DSNKIFLQSL KKDLIHYSPN SLQKFFQFNM PADLAPNDTI |
| | LPNWLKFCSV KENEKVILKK |
| 301 | LFNNFETLEN FEMQRLEKCL KFKKKPLHQK QLSQKQRGPQ |
| | STDDSKLYSL TSLQRQYKSS |
| 361 | LKSNIQKNQK LKLIIPKNNT SSSPSPLSSD DTIMSPINDY |
| | ELTEGIQSFT KNRYSNILPY |
| 421 | EHSRVKLPHS PKPPAVSEAS TTETKTDKSY PMCPVDAKNH |
| | SCKPNDYINA NYLKLTQINP |
| 481 | DFKYIATQAP LPSTMDDFWK VITLNKVKVI ISLNSDDELN |
| | LRKWDIYWNN LSYSNHTIKL |
| 541 | QNTWENICNI NGCVLRVFQV KKTAPQNDNI SQDCDLPHNG |
| | DLTSITMAVS EPFIVYQLQY |
| 601 | KNWLDSCGVD MNDIIKLHKV KNSLLFNPQS FITSLEKDVC |
| | KPDLIDDNNS ELHLDTANSS |
| 661 | PLLVHCSAGC GRTGVFVTLD FLLSILSPTT NHSNKIDVWN |
| | MTQDLIFIIV NELRKQRISM |
| 721 | VQNLTQYIAC YEALLNYFAL QKQIKNALPC |

The second was type 2C protein phosphatase PTC1 from S. cerevisiae S288C (Genbank Accession No. DAA11842.1) having the amino acid sequence of SEQ ID NO: 2:

| | |
|---|---|
| 1 | MSNHSEILER PETPYDITYR VGVAENKNSK FRRTMEDVHT |
| | YVKNFASRLD WGYFAVFDGH |
| 61 | AGIQASKWCG KHLHTIIEQN ILADETRDVR DVLNDSFLAI |
| | DEEINTKLVG NSGCTAAVCV |
| 121 | LRWELPDSVS DDSMDLAQHQ RKLYTANVGD SRIVLFRNGN |
| | SIRLTYDHKA SDTLEMQRVE |
| 181 | QAGGLIMKSR VNGMLAVTRS LGDKFFDSLV VGSPFTTSVE |
| | ITSEDKFLIL ACDGLWDVID |
| 241 | DQDACELIKD ITEPNEAAKV LVRYALENGT TDNVTVMVVF L |

The coding regions of the genes encoding PTP2 and PTC1 were amplified by PCR using chromosomal DNA of the S. cerevisiae strain FERMAX™ Gold Label (Martrex Inc., Minnesota, USA; herein abbreviated, “FG”), a well-known fermentation yeast used in the grain ethanol industry), and are represented by SEQ ID NOs: 3 and 4, below. The coding region of the PTP2 gene is shown, below, as SEQ ID NO: 3:

ATGGATCGCATAGCACAGCAATATCGTAATGGCAAAAGAGACAATAACGG
CAATAGAATGGCTTCTTCCGCTATATCGGAAAAGGGCCACATACAAGTCA
ATCAAACTAGAACACCTGGTCAAATGCCCGTCTATAGAGGTGAAACTATA
AATCTGTCTAACCTTCCCCAAAATCAAATTAAACCGTGCAAAGATTTGGA
CGACGTTAACATACGGCGGAACAACTCTAATAGGCATTCTAAAATACTTT
TACTAGATCTGTGCGCTGGCCCCAATACCAACTCATTTTTAGGCAATACC
AATGCTAAGGATATCACAGTTTTATCGTTGCCGCTACCCAGCACTTTGGT
GAAAAGGTCGAACTACCCGTTCGAGAACTTACTAAAGAATTACCTTGGAT
CTGATGAAAAGTATATTGAGTTCACAAAGATCATCAAAGATTATGATATT
TTCATTTTCAGTGATTCGTTTAGCAGAATTTCGAGTTGTTTAAAAACAAC
TTTTTGCCTCATTGAGAAGTTTAAAAAGTTCATCTGCCATTTTTTTCCAT
CTCCTTATTTGAAATTCTTTCTTCTCGAAGGCTCTCTGAATGATAGCAAG
GCCCCCTCATCAGGAAAAAATAAGAAAAATTGCATCTTGCCCAAATTGGA
TTTGAACTTGAATGTAAACTTAACTTCAAGGTCAACTTTAAATTTAAGAA
TAAACATACCTCCACCCAATGATTCAAATAAAATATTTTTACAGTCTCTG
AAAAAGGATTTAATTCATTATTCTCCTAATTCTTTGCAAAAGTTTTTCCA
ATTCAATATGCCTGCTGACTTAGCACCTAACGACACGATTTTACCGAATT
GGCTAAAATTCTGCTCCGTAAAAGAAAATGAAAAGGTAATATTAAAGAAA
CTCTTTAACAATTTTGAAACTTTAGAAAATTTTGAAATGCAAAGATTAGA
GAAATGCCTGAAATTCAAGAAAAAGCCTTTACATCAAAAGCAGCTATCAC
AAAAGCAGAGGGGTCCGCAATCCACGGATGATTCAAAATTATATTCTTTA
ACTAGTTTGCAACGACAGTATAAAAGTTCTTTGAAAAGCAACATACAGAA
AAATCAAAACCTAAAATTAATTATACCAAAAAACAACACATCTTCTTCGC
CATCACCATTATCTTCCGATGATACTATAATGACACCAATAAATGATTAC
GAACTTACTGAAGGAATTCAGTCTTTTACTAAGAATAGATATTCTAATAT
CTTACCTTACGAACATTCAAGAGTAAAGTTACCTCACTCCCCGAAACCAC
CTGCAGTTTCTGAAGCATCCACAACCGAAACTAAAACAGATAAGTCATAT
CCGATGTGTCCCGTAGATGCAAAAAACCACTCCTGCAAACCGAACGACTA
TATCAATGCGAACTATTTGAAGCTCACGCAAATTAATCCTGATTTCAAGT
ATATTGCTACCCAAGCTCCGCTTCCTTCTACGATGGATGATTTTTGGAAG
GTTATTACTTTAAATAAAGTTAAAGTAATAATATCATTGAATTCTGACGA
TGAATTGAATTTAAGAAAATGGGATATTTACTGGAATAATCTATCATATT
CCAACCACACTATCAAACTTCAAAACACCTGGGAGAATATTTGCAATATT
AACGGCTGTGTTCTCAGAGTCTTTCAAGTCAAGAAAACAGCTCCACAAAA
TGATAATATCAGTCAAGATTGTGACCTTCCGCATAATGGTGACCTTACTT
CCACTACCATGGCTGTATCCGAGCCGTTTATTGTTTACCAATTACAATAC
AAGAATTGGTTAGATTCATGCGGCGTAGATATGAATGACATCATTAAACT
ACACAAAGTCAAAAATTCGTTATTGTTTAACCCGCAAAGTTTTATTACAA
GCCTCGAAAAGGATGTTTGCAAGCCTGATTTGATAGATGATAATAATAGT
GATTTACATCTCGATACAGCAAATTCATCGCCGCTATTAGTCCATTGTTC
TGCAGGGTGTGGAAGAACAGGTGTTTTCGTTACCTTGGATTTCCTGCTAA
GTATTCTTTCACCTACAACAAATCACTCAAATAAGATTGATGTTTGGAAT
ATGACTCAGGACCTTATCTTTATCATAGTGAATGAATTAAGAAAGCAAAG
GATTTCAATGGTACAGAATCTAACTCAATATATCGCTTGTTATGAGGCAT
TATTAAATTATTTTGCCCTGCAAAAGCAGATAAAGAACGCGTTACCTTGT
TAA

The coding region of the PTC1 gene is shown, below, as SEQ ID NO: 4:

ATGAGTAATCATTCTGAAATCTTAGAAAGGCCAGAAACACCATATGACAT
AACTTATAGAGTAGGTGTGGCGGAAAATAAAAACTCGAAATTTCGGAGGA
CAATGGAAGATGTTCATACGTATGTTAAAAACTTTGCTTCAAGATTAGAT
TGGGGATATTTCGCGGTGTTTGATGGACATGCTGGGATTCAGGCCTCCAA
ATGGTGTGGTAAACATCTTCATACAATTATAGAGCAAAACATTTTGGCAG
ATGAAACACGAGATGTTAGAGATGTATTGAACGATTCATTCCTAGCCATT
GACGAAGAAATTAATACAAAACTTGTAGGAAATAGTGGATGTACTGCTGC
TGTTTGCGTATTACGTTGGGAGCTTCCGGATTCAGTTTCTGATGATTCAA
TGGATTTAGCCCAACACCAAAGAAAGTTATATACAGCAAATGTTGGTGAT
TCTCGAATAGTATTGTTTAGAAACGGGAACAGCATAAGACTGACTTATGA
TCATAAAGCATCTGACACTTTGGAGATGCAGAGAGTTGAACAAGCAGGTG
GCCTGATAATGAAAAGTCGTGTAAATGGTATGCTGGCGGTGACGAGATCG
TTAGGGGATAAATTTTTTGATAGTTTAGTAGTGGGCAGCCCATTTACCAC
GAGCGTAGAAATAACTTCTGAGGACAAATTTTTAATCCTAGCGTGTGATG
GATTATGGGATGTTATTGATGATCAAGATGCATGCGAATTAATCAAGGAT
ATTACTGAACCTAATGAAGCTGCAAAAGTCTTGGTTAGATATGCTTTGGA
AAATGGCACAACAGATAATGTAACGGTCATGGTTGTCTTCCTCTAA

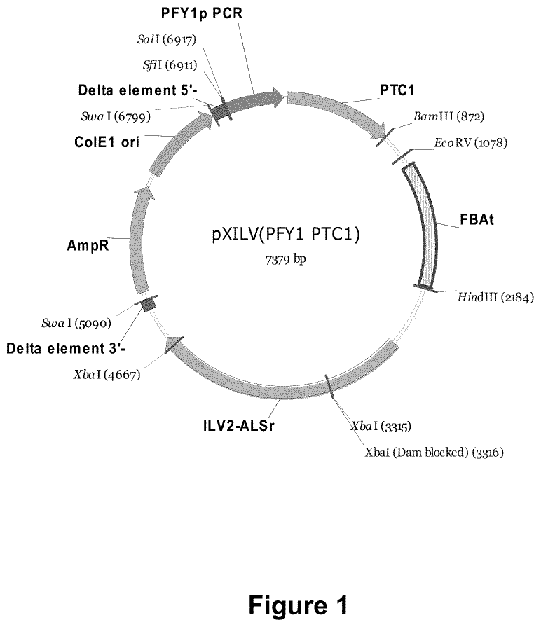

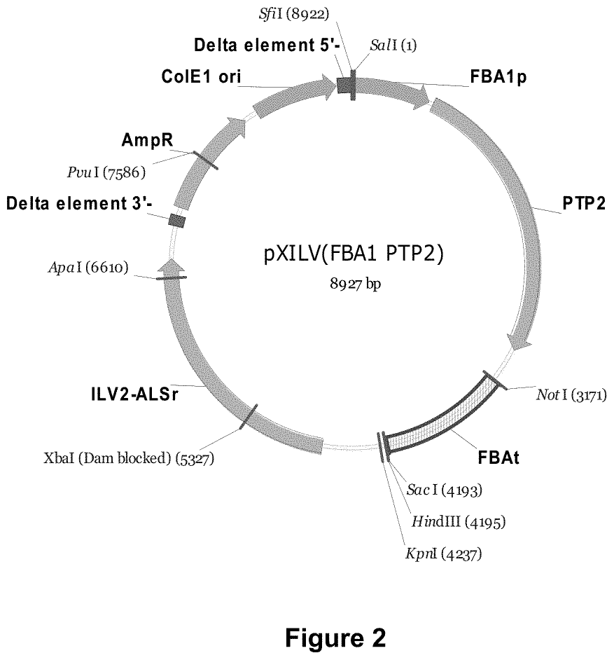

The genes were synthesized and cloned into expression vectors under the control of the FBA1 promoter and FBA1 terminator. Maps of the expression vectors are shown in FIGS. 1 and 2. Similar constructs are expected to produce similar results.

The FBA1 promoter region used for over-expression is represented by SEQ ID NO:5:

GACGATGAGGAAGAGGAGGCTGCGTTTGACGACGAAGAGGATGATAATGA
GGAAGAAGAAGAAGAAGAGGACGCGGATGAAGAGAACGCCTCTCGTCTAA
GAAATTTAAAAAGAGAAGGAGCAGCAATGTACAGAGAAGAGGAAGAAGAA
GAAAAAGATAGGAGCGAGACAAAAAGAAGAAGGGTTGCGGTCATCGAGGA
CGACGAAGACGAGGATTAGAGGAGACGTTACTTTGTTTATATATATTAGT
ATGTACAATCGCAAAGAAATGGAGTGATGACATGTTGTAGTATTTAGTAT
GAGGTTACTGTGTGGGAGATTTTACCATGATTTTTGGCGAGAACACGCCA
TGAAATGTCTTTGTACGAAAATCATTACCCGCATTAATATTTTTTTTCTT
TTTAAAGCTCAGTTGACCCTTTCTCATTCCCTTCTTAAAACAACTGTGTG
ATCCTTG

The FBA1 terminator is represented by SEQ ID NO: 6:

GTTAATTCAAATTAATTGATATAGTTTTTTAATGAGTATTGAATCTGTTT
AGAAATAATGGAATATTATTTTTATTTATTTATTTATATTATTGGTCGGC
TCTTTTCTTCTGAAGGTCAATGACAAAATGATATGAAGGAAATAATGATT
TCTAAAATTTTACAACGTAAGATATTTTTACAAAAGCCTAGCTCATCTTT
TGTCATGCACTATTTTACTCACGCTTGAAATTAACGGCCAGTCCACTGCG
GAGTCATTTCAAAGTCATCCTAATCGATCTATCGTTTTTGATAGCTCATT
TTGGAGTTCGCGATTGTCTTCTGTTATTCACAACTGTTTTAATTTTTATT
TCATTCTGGAACTCTTCGAGTTCTTTGTAAAGTCTTTCATAGTAGCTTAC
TTTATCCTCCAACATATTTAACTTCATGTCAATTTCGGCTCTTAAATTTT
CCACATCATCAAGTTCAACATCATCTTTTAACTTGAATTTATTCTCTAGC
TCTTCCAACCAAGCCTCATTGCTCCTTGATTTACTGGTGAAAAGTGATAC
ACTTTGCGCGCAATCCAGGTCAAAACTTTCCTGCAAAGAATTCACCAATT
TCTCGACATCATAGTACAATTTGTTTTGTTCTCCCATCACAATTTAATAT
ACCTGATGGATTCTTATGAAGCGCTGGGTAATGGACGTGTCACTCTACTT
CGCCTTTTTCCCTACTCCTTTTAGTACGGAAGACAATGCTAATAAATAAG
AGGGTAATAATAATATTATTAATCGGCAAAAAAGATTAAACGCCAAGCGT
TTAATTATCAGAAAGCAAACGTCGTACCAATCCTTGAATGCTTCCCAATT
GTATATTAAGAGTCATCACAGCAACATATTCTTGTTATTAAATTAATTAT
TATTGATTTTTGATATTGTATAAAAAAACCAAATATGTATAAAAAAAGTG
AATAAAAAATACCAAGTATGGAGAAATATATTAGAAGTCTATACGTTAAA

For selection in yeast, a chlorimuron-resistant form of the acetolactate synthase gene (ILV2) from S. cerevisiae was used, the gene having a single nucleotide mutation resulting in a single amino acid change that confers insensitivity to chlorimuron. The sequence of the gene is shown, below, as SEQ ID NO: 7:

TAGGTGACAAACGCCTAGCCGCCGGAGCCTGCCGGTACCGGCTTGGCTTC
AGTTGCTGATCTCGGCGCGGAAAAATCAGCGCCCCACGCCAAAAGGTTCG
TATTTTTTCTTTTTTTTTCTAATCTTCCATCTATTCGGTAGCGATGATTC
ATTTCTCTGAAAAAAAAAAAAAAAAAAAAAAATGAAAAAGAATATTTTTT
TGATGAACTTGTATTTCTCTTATCTGGTTGATATATATGCTATCATTTAT
TTTCTTATCAAGTTTCCAAATTTCTAATCCTTTCTCCACCATCCCTAATT
AATAATTCAGATCTACGTCACACCGTAATTTGTATTGTTTTTTTCCTTCA
TTGTCTAAAACCGAAGAATTCATCAGCCACAGTTACTAGTTCATTTGAAG
CGAAATTACACACATTTTCCCTGTTACAATAGAAAGTATTTTACAAAATC
TAAACCCTTTGAGCTAAGAGGAGATAAATACAACAGAATCAATTTTCAAA
TGATCAGACAATCTACGCTAAAAAACTTCGCTATTAAGCGTTGCTTTCAA
CATATAGCATACCGCAACACACCTGCCATGAGATCAGTAGCTCTCGCGCA
GCGCTTTTATAGTTCGTCTTCCCGTTATTACAGTGCGTCTCCATTACCAG
CCTCTAAAAGGCCAGAGCCTGCTCCAAGTTTCAATGTTGATCCATTAGAA
CAGCCCGCTGAACCTTCAAAATTGGCTAAGAAACTACGCGCTGAGCCTGA
CATGGATACCTCTTTCGTCGGTTTAACTGGTGGTCAAATATTTAACGAAA
TGATGTCCAGACAAAACGTTGATACTGTATTTGGTTATCCAGGTGGTGCT
ATCCTACCTGTTTACGATGCCATTCATAACAGTGATAAATTCAACTTCGT
TCTTCCAAAACACGAACAAGGTGCCGGTCACATGGCAGAAGGCTACGCCA
GAGCTTCTGGTAAACCAGGTGTTGTCTTGGTTACTTCTGGGCCAGGTGCC
ACCAATGTCGTTACTCCAATGGCAGATGCCTTTGCAGACGGGATTCCAAT
GGTTGTCTTTACAGGGCAAGTCTCAACTAGTGCTATCGGTACTGATGCTT
TCCAAGAGGCTGACGTCGTTGGTATTTCTAGATCTTGTACGAAATGGAAT
GTCATGGTCAAGTCCGTGGAAGAATTGCCATTGCGTATTAACGAGGCTTT
TGAAATTGCCACGAGCGGTAGACCGGGACCAGTCTTGGTCGATTTACCAA
AGGATGTTACAGCAGCTATCTTAAGAAATCCAATTCCAACAAAAACAACT
CTTCCATCAAACGCACTAAACCAATTAACCAGTCGCGCACAAGATGAATT
TGTCATGCAAAGTATCAATAAAGCAGCAGATTTGATCAACTTGGCAAAGA
AACCTGTCTTATACGTCGGTGCTGGTATTTTAAACCATGCAGATGGTCCA
AGATTACTAAAAGAATTAAGTGACCGTGCTCAAATACCTGTCACCACTAC
TTTACAAGGTTTAGGTTCATTCGACCAAGAAGATCCAAAATCATTGGATA
TGCTTGGTATGCACGGTTGTGCTACTGCCAACCTGGCAGTGCAAAATGCC
GACTTGATAATTGCAGTTGGTGCTAGATTCGACGACCGTGTCACTGGTAA
TATTTCTAAATTCGCTCCAGAAGCTCGTCGTGCAGCTGCCGAGGGTAGAG
GTGGTATTATTCATTTCGAGGTTAGTCCAAAAAACATAAACAAGGTTGTT
CAAACTCAAATAGCAGTGGAAGGTGATGCTACGACCAATCTGGGCAAAAT
GATGTCAAAGATTTTCCCAGTTAAGGAGAGGTCTGAATGGTTTGCTCAAA
TAAATAAATGGAAGAAGGAATACCCATACGCTTATATGGAGGAGACTCCA
GGATCTAAAATTAAACCACAGACGGTTATAAAGAAACTATCCAAGGTTGC
CAACGACACAGGAAGACATGTCATTGTTACAACGGGTGTGGGGCAACATC
AAATGTGGGCTGCTCAACACTGGACATGGAGAAATCCACATACTTTCATC
ACATCAGGTGGTTTAGGTACGATGGGTTACGGTCTCCCTGCCGCCATCGG
TGCTCAAGTTGCAAAGCCAGAATCTTTGGTTATTGACATTGATGGTGACG
CATCCTTTAACATGACTCTAACGGAATTGAGTTCTGCCGTTCAAGCTGGT
ACTCCAGTGAAGATTTTGATTTTGAACAATGAAGAGCAAGGTATGGTTAC
TCAATGGCAATCCCTGTTCTACGAACATCGTTATTCCCACACACATCAAT
TGAACCCTGATTTCATAAAACTAGCGGAGGCTATGGGTTTAAAAGGTTTA
AGAGTCAAGAAGCAAGAGGAATTGGACGCTAAGTTGAAAGAATTCGTTTC
TACCAAGGGCCCAGTTTTGCTTGAAGTGGAAGTTGATAAAAAAGTTCCTG
TTTTGCCAATGGTGGCAGGTGGTAGCGGTCTAGACGAGTTCATAAATTTT
GACCCAGAAGTTGAAAGACAACAGACTGAATTACGTCATAAGCGTACAGG
CGGTAAGCACTGAATTTCAAAAACATTTATTTCAAAAGCATTTTCAGTAA
AAAATGCAGACTTTATTATTATTTAATCGTGCTTCTTATATATGACATTC
TACCAAATCGGTAGTCATGTATATTTTTTTCGTATATACTTTATATATTT
TTTTCTAAAAAACTAATGACGGCTAAAATTAAGTCATAGATGAATAATAA
GTTCAATTCAAGTGAGTTGGTAGTATTTGATAAATCTCGCGCCGAAGCTG
AAGTGCAAGGATTGATAATG

Linear PTP2 and PTC expression cassettes were generated by SwaI digestion and used to transform yeast strain FERMAX™ Gold (Martrex Inc., Minnesota, USA; herein abbreviated, “FG”) to chlorimuron resistance. Selection was done on SC_chl_30 plates (Yeast nitrogen base w/o amino acids 6.7 g/l, glucose 20 g/l, chlorimuron 30 mg/1, Bacto-agar 20 g/l). Several transformants were picked from each transformation, purified by sub-cloning on the same medium.

### Example 3. Alcohol Production Using Yeast that Over-Express Protein Phosphatase

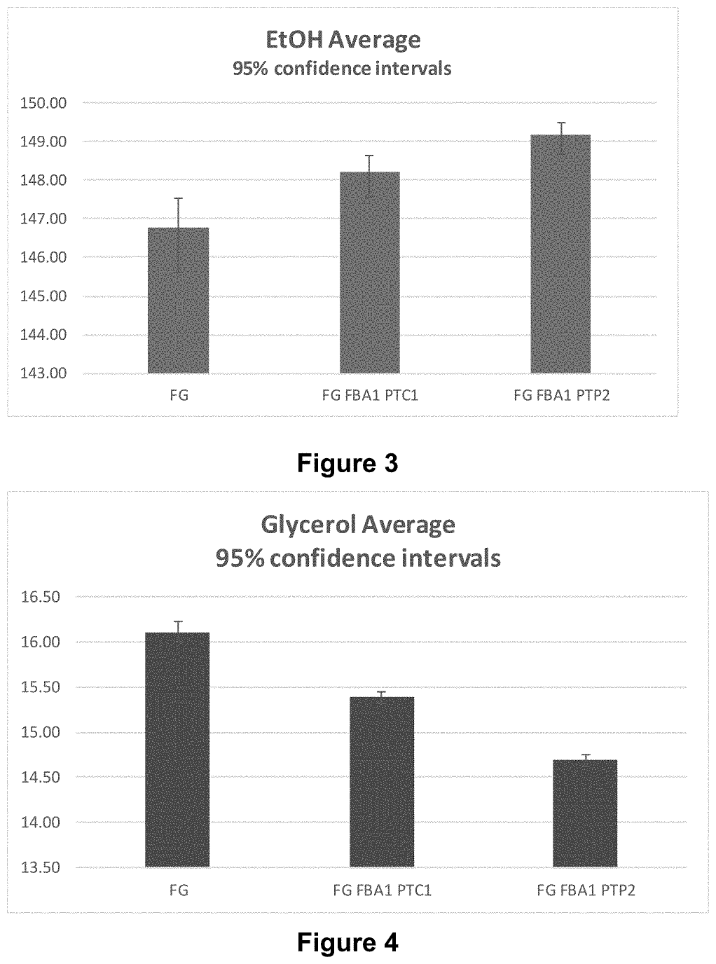

Strains over-expressing protein phosphatase were tested in a MTP-based fermentation assay described above. 89 wells that were intentionally randomly located on the plate to avoid assay variations (especially those caused by so called “edge effects”), were inoculated with yeast of each strain. Samples from the end of fermentation were analyzed by HPLC. The results are summarized in Tables 1 and 2 and FIGS. 3 and 4.

TABLE 1
Amount of ethanol produced by modified cells

| | | | | 95% | 90% | |
|---|---|---|---|---|---|---|
| Strain | N | Average | % of FG | conf. | conf. | StDev |
| FG | 9 | 146.77 | 100.0 | 0.76 | 0.64 | 1.17 |
| FG FBA1 PTC1 | 9 | 148.22 | 101.0 | 0.41 | 0.35 | 0.63 |
| FG FBA1 PTP2 | 8 | 149.17 | 101.6 | 0.34 | 0.28 | 0.49 |

TABLE 2
Amount of glycerol produced by modified cells

| | | | | 95% | 90% | |
|---|---|---|---|---|---|---|
| Strain | N | Average | % of FG | conf | conf | StDev |
| FG | 9 | 16.11 | 100.0 | 0.11 | 0.09 | 0.17 |
| FG FBA1 PTC1 | 9 | 15.39 | 95.5 | 0.07 | 0.06 | 0.10 |
| FG FBA1 PTP2 | 8 | 14.69 | 91.1 | 0.07 | 0.06 | 0.11 |

Over-expression of PTC1 resulted in a 1.0% increase in ethanol production and 4.5% decrease in glycerol production. Over-expression of PTP2 resulted in a 1.6% increase in ethanol production and 9.9% decrease in glycerol production. The number of replicates (N), within 90 and 95% confidence levels, and standard deviation values (StDev), are indicated.

What is claimed is:

1. Modified yeast cells derived from parental yeast cells, the modified cells comprising a genetic alteration that causes the modified cells to produce an increased amount of protein phosphatase polypeptides associated with the high-osmolarity glycerol pathway compared to the parental cells, wherein the modified cells produce during fermentation an increased amount of ethanol and/or a decreased amount of glycerol compared to the amount of ethanol and glycerol produced by the parental cells under equivalent fermentation conditions.

2. The modified cells of claim 1, wherein the genetic alteration comprises the introduction into the parental cells of a nucleic acid capable of directing the expression of a protein phosphatase polypeptide to a level above that of the parental cell grown under equivalent conditions.

3. The modified cells of claim 1, wherein the genetic alteration comprises the introduction of an expression cassette for expressing a protein phosphatase polypeptide.

4. The modified cells of any of claims 1-3, wherein the protein phosphatase is PTC1 or PTP2.

5. The modified cells of any of claims 1-4, wherein the cells further comprising one or more genes of the phosphoketolase pathway.

6. The modified cells of claim 5, wherein the genes of the phosphoketolase pathway are selected from the group consisting of phosphoketolase, phosphotransacetylase and acetylating acetyl dehydrogenase.

7. The modified cells of any of claims 1-6, wherein the amount of increase in the expression of the protein phosphatase polypeptide is at least about 200% compared to the level expression in the parental cells grown under equivalent conditions.

8. The modified cells of any of claims 1-7, wherein the amount of increase in the production of mRNA encoding the protein phosphatase polypeptide is at least about 400% compared to the level in the parental cells grown under equivalent conditions.

9. The modified cells of any of claims 1-8, wherein the cells further comprise an exogenous gene encoding a carbohydrate processing enzyme.

10. The modified cells of any of claims 1-9, further comprising an alteration in the glycerol pathway and/or the acetyl-CoA pathway.

11. The modified cells of any of claims 1-10, further comprising an alternative pathway for making ethanol.

12. The modified cells of any of claims 1-11, wherein the cells are of a Saccharomyces spp.

13. A method for decreasing the production of acetate from yeast cells grown on a carbohydrate substrate, comprising: introducing into parental yeast cells a genetic alteration that increases the production of protein phosphatase polypeptides compared to the amount produced in the parental cells.

14. The method of claim 13, wherein the cells having the introduced genetic alteration are the modified cells are the cells of any of claims 1-12.

15. The method of claim 13 or 14, wherein the decrease in acetate production is at least 10%, at least 15%, at least 20%, or at least 25%.

16. The method of any of claims 13-15, wherein protein phosphatase polypeptides are over-expressed by at least 200%.

17. The method of any of claims 12-16, wherein protein phosphatase polypeptides are over-expressed by at least 15-fold.

18. The method of any of claims 12-17, wherein the carbohydrate substrate is present in an industrial medium.
